(19) United States
(12) Patent Application Publication
(10) Pub. No.: US 2016/0274085 A1
(43) Pub. Date: Sep. 22, 2016

(54) 3D CELL CULTURE AND EX VIVO DRUG TESTING METHODS

(71) Applicant: MOLECULAR RESPONSE, LLC, San Diego, CA (US)

(72) Inventors: Praveen NAIR, San Diego, CA (US); Cyrus Mirsaidi, San Diego, CA (US); Tommy Broudy, San Diego, CA (US)

(21) Appl. No.: 15/036,808

(22) PCT Filed: Nov. 13, 2014
(86) PCT No.: PCT/US2014/065549

§ 371 Date: May 14, 2016

Related U.S. Application Data
(60) Provisional application No. 61/905,040, filed on Nov. 15, 2013.

(51) Int. Cl. G01N 33/50

(52) U.S. Cl. CPC G01N 33/5011; G01N 33/5088; G01N 2510/00

(57) ABSTRACT

Provided herein are methods for testing proliferative responses of a drug on patient-derived tumor cells; the method comprising obtaining cells from biopsy or tumor resection material; culturing the cells on a 3D extracellular matrix (ECM); treating the cells in ECM with a drug; subjecting the treated cells to high-content (HC) imaging; and evaluating the HC imaging of the treated cells; thereby testing the proliferative responses of the drug on the patient-derived tumor cells. In some embodiments, the methods disclosed herein comprise obtaining cells from biopsy or tumor resection material; xenografting the cells into an animal model (patient-derived xenograft; PDX) for tumor formation; and obtaining tumor cells from the animal.

The present application claims priority to provisional U.S. patent application No. 61/905,040, filed Nov. 15, 2013, which is hereby incorporated in its entirety including all tables, figures, and claims.

### FIELD OF THE INVENTION

The present invention relates to the use of 3D (3-dimensional) cell culture technology in methods for ex vivo drug testing, wherein cells acquire a natural 3D phenotype resulting in the capability for increased cell proliferation, differentiation, and function.

### BACKGROUND OF THE DISCLOSURE

The current trend in drug discovery and disease-related research is moving away from the use of non-human primate animal models. This trend was underscored recently by the significant reduction of the use of chimpanzees in NIH research. Moreover, it is now known that conventional 2D (2-dimensional) cell culture methods do not accurately represent the real 3D world in disease progression, drug testing and/or biochemical and physiological research. Therefore, advanced in vitro platform-based technologies, utilizing sophisticated scaffolds and extracellular matrices to support cell growth, have been and are continuing to be developed. This is because 3D cell culture platform-based methods allow cells to acquire a natural 3D phenotype and permit increased cell proliferation, differentiation, and function.

However, 3D cell culture techniques have current limitations including poor reproducibility and stability, complexity of components, difficulty with scaling up or down, and/or a need for improvement of physiological substrates. In particular, anti-proliferative predictive assays of oncology drug candidates using patient-derived tumor cells, tested in a 3D growth environment, produce unpredictable results due to high heterogeneity, low proliferation, and/or low availability of well-characterized samples.

Accordingly, there is a need in the art to overcome such limitations. The present methods utilize starting material from a high quality source of quantified tumor biobank samples and/or samples retrospectively annotated and prospectively procured, optimized 3D growth conditions, and sophisticated techniques for high content imaging and/or subpopulation analysis.

3D cell culture methods and related technology and apparatuses may be found, for example, in US Publication Nos. 20110143960; and 20120309016; however, none of these references and/or corresponding counterparts discloses the embodiments of the present invention.

All documents and references cited herein and in the referenced patent documents, are hereby incorporated herein by reference.

### SUMMARY OF THE INVENTION

The present inventors have developed a highly sensitive 3D spheroid primary culture platform comprising 3D spheroid cell (primary tumor or patient-derived tumor cells) overlay on ECMs, drug treatment paradigms, ex vivo proliferation endpoints, and high content cellular imaging, data analysis and interpretation for actionable insight. In particular, the inventors have developed innovative methodology utilizing retrospectively annotated and prospectively procured samples and/or a bio-bank of tumor biopsy samples with associated molecular and treatment data, optimized conditions to grow the samples as 3D spheroids, high content imaging as a read out, and optimized labeling for subpopulation analysis. Accordingly, predictive proliferative assays developed from such methodology reliably screen drug candidates for efficacy and/or mechanism of action.

Disclosed herein is a method of testing proliferative responses of a drug on patient-derived tumor cells; the method comprising, obtaining cells from biopsy or tumor resection material; culturing the cells on a 3D extracellular matrix (ECM); treating the cells in ECM with a drug; subjecting the treated cells to high-content (HC) imaging; and evaluating the HC imaging of the treated cells; thereby testing the proliferative responses of the drug on the patient-derived tumor cells.

In another embodiment, the treated cells are in the formation of a tumor spheroid.

In another embodiment, the biopsy or tumor resection material is from a biobank.

In another embodiment, the patient-derived tumor cells are primary tumor cells (PTCs).

In another embodiment, the patient-derived tumor cells are selected from the group consisting of breast cancer cells, prostate cancer cells, non-small cell lung cancer cells, ovarian cancer cells, melanoma cells, and pancreatic cancer cells.

In another embodiment, the drug is selected from the group consisting of small molecule drugs, kinase inhibitors, macromolecules, and a combination thereof.

In another embodiment, the proliferative responses are selected from the group consisting of EdU incorporation, LIVE-DEAD cell counts, colony formation, and a combination thereof.

In another embodiment, the treated cells are evaluated by techniques selected from the group consisting of proliferation, colony morphology, apoptosis, and a combination thereof.

Also disclosed herein is a method of testing proliferative responses of a drug on patient-derived tumor cells; the method comprising obtaining cells from biopsy or tumor resection material; xenografting the cells into a mouse for tumor formation; obtaining tumor cells from the mouse; culturing the tumor cells on a 3D extracellular matrix (ECM); treating the tumor cells in ECM with a drug; subjecting the treated tumor cells to high-content (HC) imaging; and evaluating the HC imaging of the treated cells; thereby testing the proliferative responses of the drug on the patient-derived tumor cells.

In another embodiment, the treated tumor cells are in the formation of a tumor spheroid.

In another embodiment, the biopsy or tumor resection material is from a biobank.

In another embodiment, the patient-derived tumor cells are primary tumor cells (PTCs).

In another embodiment, the patient-derived tumor cells are selected from the group consisting of breast cancer cells, prostate cancer cells, non-small cell lung cancer cells, ovarian cancer cells, melanoma cells, and pancreatic cancer cells.

In another embodiment, the drug is selected from the group consisting of small molecule drugs, kinase inhibitors, macromolecules, and a combination thereof.

In another embodiment, the proliferative responses are selected from the group consisting of EdU incorporation, LIVE-DEAD cell counts, colony formation, and a combination thereof.

In another embodiment, the treated tumor cells are evaluated by techniques selected from the group consisting of proliferation, colony morphology, apoptosis, and a combination thereof.

In other embodiments, the methods in the preceding paragraphs may additionally incorporate any of the preceding or subsequent disclosed embodiments.

The Summary of the Invention is not intended to define the claims nor is it intended to limit the scope of the invention in any manner

Other features and advantages of the invention will be apparent from the following Drawings, Detailed Description, and the Claims.

### BRIEF DESCRIPTION OF THE DRAWINGS

FIG. 1A-B shows the schema of the use of patient-derived xenografts (PDX) for harvesting cells for ex vivo proliferation assays.

FIG. 2A-C: FIG. 2A shows z-stacking of spheroids followed by maximum projections to capture cell images. FIG. 2B shows images captured by DAPI and EdU channels. FIG. 2C shows drug candidate comparison data from EdU MFI across multiple plates.

FIG. 3A-B: FIG. 3A shows images captured of spheroid morphology using 2 chemotherapeutic drug candidates. FIG. 3B shows spheroid size data for treated cells.

FIG. 4 shows images and data for apoptosis induction of straurosporine-treated PTCs.

FIG. 5A-C: FIG. 5A shows images of DAPI, EdU, and CK staining. FIG. 5B-C shows proliferation data in subsets of treated cells.

### DETAILED DESCRIPTION OF THE EMBODIMENTS

Methods for 3D spheroid cell culture and/or ex vivo proliferation assays are disclosed herein. Cryopreserved or fresh primary tumor cells (PTCs) or patient-derived tumor cells are resurrected via extracellular matrices (ECMs) to gain re-entry into a proliferative state. The 3D spheroid cell culture is subjected to treatment with a single drug or drug candidate or agent and/or combinations of drug candidates or agents. Highly sensitive and finely accurate state of the art high content imaging elucidates multiple endpoints such as proliferation state, apoptosis, morphology, and/or surface markers.

3D cell culture techniques are known and readily available to the artisan. Such techniques are continuing to develop and are becoming more and more relevant to human and animal physiology. For example, Haycock, J W reviews 3D techniques and approaches in Methods Mol. Biol. 2011; 695:1-15.

In one embodiment of the invention disclosed herein, the present inventors have helped develop and have exclusive access to a companion biobank (retrospectively annotated and prospectively procured) featuring matched DNA, RNA, and protein from cryopreserved cells. The biobank was created and continuously expanded from cells obtained from patients' tumor biopsies or resections which were then cryopreserved as live biologically intact samples. Biobank data of tumor cells include, without limitation, data relating to DNA (mutations, copy number, translocation), RNA (gene expression, splice variation), and protein (antigen detection, and size variation). Such data provides a high level of quality assurance based on its opportunities for DNA, RNA and/or protein sampling, patient selection paradigms, cell screening, phenotyping, and/or discovery of new markers. Accordingly, the biobank offers a full suite of molecular testing capabilities to screen for tumors with molecular features of interest.

Ex vivo proliferation assays of the present methods provide an effective way to measure anti-proliferation activities of anti-cancer agents in physiologically relevant environments. Tumor cells derived from biopsy or resection material are used as single cell suspension or in cell culture and conditioned to form spheroids in vitro to mimic the tumor environment. Proliferation of individual cells is measured at the end of the treatment with a drug candidate(s) using high content imaging to measure anti-proliferative potential for the drug candidate(s).

In some embodiments, the present methods comprise the use of an animal xenograft such as a murine animal xenograft. Fresh or banked patient-derived tumor cells are injected into, for example, an immunocompromized mouse for development of patient-derived xenografts (PDX). Harvested cells are plated to form spheroids for ex vivo drug treatment. Cell proliferation is measured by an EdU incorporation assay to assess the effects of a drug candidate(s) or agent(s) such as chemotherapy (e.g., cisplatin, doxorubicin) agents.

In some embodiments, the present methods comprise any one or more of transport media, enzyme digestion, cryoprotectants, thawing methods, clean up protocols, viability assessments, conditioning techniques, control fibroblasts, media selection, ECMs, types of spheroids, imaging techniques, segmentation, predictive assays, and/or sub-population analysis. In certain embodiments, the present methods comprise the use of cryopreserved or fresh primary tumor cells (PTCs), resurrection and/or re-entry into proliferative state, single and/or combinations of drug treatment paradigms, and/or high content imaging for analysis of proliferation, apoptosis, morphology, and/or surface markers.

In another embodiment, the present methods comprise proliferation assays for tumor spheroids, subpopulation analyses, and/or colony morphology assays. In such embodiments, proliferating cells incorporate EdU, cells are imaged and quantified using ImageXpress® Micro (Molecular Devices, Sunnyvale, Calif.). With the use of the methods disclosed herein, tumor spheroids reliably resemble the tumor microstructure and by staining for specific markers, heterogeneous cell populations can be detected and analyzed. In addition, primary tumor cell (PTC) samples are attractive models because they reflect the heterogeneity of cancer and may better predict response to anticancer agents.

Primary tumor cells (PTCs) are capable of forming spheroids. Spheroids are complicated structures and require z-stacking followed by maximum projection to capture cell images via high content imaging. After the z-stacking, the images are processed using various algorithms to characterize the cells. DAPI (channel 1) is used to identify valid objects. Edu (channel 2) is used to identify a population of cells incorporating DNA. In this particular instance, the present inventors assigned a white mask to identify EdU negative (−) cells and a red mask to identify EdU positive (+) cells. However, representative masks/gates can be assigned to positive and negative cells to do population analysis. Intensity for each pixel for each channel is recorded and integrated to obtain the total intensity. Total intensity divided by total pixels gives mean fluorescent intensity (MFI) for that channel. Integrated mean fluorescent intensity of multiple cells divided by total cells per well gives mean of mean fluorescent intensity. To normalize the variability of cell numbers between wells the mean of mean fluorescent intensities of EdU signals are used to assess in vitro drug effects. EdU mean fluorescent intensities are used to compare the drug effects across multiple plates.

Spheroid morphology or colony morphology can be used as a measure of drug effects on patient-derived tumor cells growing as spheroids. In this manner, size of spheroid or the tumor outgrowth is used to assess the effect of chemotherapeutic agents. Other end points can also be used to evaluate effects of drug candidates. For example, apoptosis induction in the cells is measured using CellEvent™ and quantified using high content imaging.

Sub-population analysis of tumor tissue involves a number of approaches. By combining EdU incorporation assay with staining for tumor specific markers, the effect of drug candidates in specific cell populations can be assessed. Cytokeratin (CK) is used as a marker for epithelial cells (mostly tumor cells). Fluorescent images of spheroids are analyzed using image cytometry algorithms to look at the proliferation in subsets of cells and analyzed for drug effects on the cell populations of interest.

The methods disclosed herein leverage access to a high quality and uniquely quantified primary tumor bank, and thus garnering a high level of expertise, knowledge, and information in clinical trial designs. Hypothesis driven study designs involve ex vivo treatment studies which assess marker frequency, e.g., marker (+) versus marker (−) tumors, through single drug or combination of drug paradigms. Hypothesis development study designs identify responder and non-responder populations and/or marker discovery for clinical trials. Accordingly, the present methods comprise subpopulation analysis providing robust sensitivity and accurate quantitation of primary tumor cells in 3D matrix measured by state of the art high content imaging with multi-channel and multi-endpoints (e.g., nuclear stain (DAPI, Hoechst), epithelial cell, cytokeratin (CK), proliferating cell (EdU), and/or open channel for additional markers).

3D cell overlay on extracellular matrices comprises treatment in triplicate in a 60 well format (excluding outer wells; media in the outer wells). The study design utilizing 3D cell overlay with primary tumor cells or PTCs comprises a proliferation endpoint. Study variables include, but are not limited to, dose range, dosing frequency, exposure time, duration of assay, combination of agents, accounting for order of addition, timing of addition, relative potency, and/or concentration. Study design variables include, but are not limited to, drug mechanism of action (MOA), protein kinase (PK), compound stability, clinical plan, target expression, and/or cell activity.

The present methods comprise image analysis of 3D spheroid primary culture model platform. In some embodiments, the methods use ImageXpress® Micro (Molecular Devices, CA) to capture high content images of PTCs and/or MetaXpress® 3.1.0.81 to analyze images. Custom package “multi-wavelength cell scoring” may be used to determine DAPI signals in channel 1 and EdU signals in channel 2 (Cy5 channel). The algorithm comprises DAPI (channel 1) to identify valid objects (denominator) and EdU (channel 2) to identify populations of cells incorporating DNA (numerator). Intensity for each pixel and for each channel is recorded and integrated to obtain the total intensity. Total intensity is divided by total pixels to give the mean fluorescent intensity (MFI) for that channel. Integrated MFI of multiple cells divided by total cells per well give the mean of MFI. To normalize the variability of cell numbers between wells the mean of the MFIs of EdU signals is used.

The methods disclosed herein are used with patient-derived tumor cells such as primary tumor cells (PTCs). Patient-derived tumor cells can be cells of any tumor type such as breast cancer cells, prostate cancer cells, non-small cell lung cancer cells, ovarian cancer cells, melanoma cells, and pancreatic cancer cells.

The methods disclosed herein measure and evaluate proliferative responses including without limitation, EdU incorporation, LIVE-DEAD cell counts, colony formation, and/or a combination thereof. The methods disclosed herein also use evaluating techniques including, without limitation, proliferation, colony morphology, apoptosis, and/or a combination thereof.

Clinical trials have been plagued by the inclusion of unselected patient populations not benefiting from any given tested therapy. This is particularly true in the cancer field. It is therefore becoming more and more acceptable to perform diagnostics and/or selection or screening assays measuring and/or analyzing and/or discovering biomarkers and/or other endpoints. Therefore, the methodology disclosed herein provides a mechanism through which clinical trial therapies can enrich patients that are selectively dependent on the targeted therapeutic. As such, a patient selection strategy allows one to improve the clinical benefit of the targeted therapeutic and excludes patients that would not benefit from treatment of the targeted therapeutic.

Drugs and/or candidate drugs and/or drug agents include but are not limited to biological molecules such as nucleic acid and amino acid macromolecules, kinase inhibitors, chemically synthesized active substance small molecules, synthesized amino acid macromolecules that are linked with small molecules and/or a combination thereof. Small molecules are generally known to be low molecular weight compounds which often function as an enzyme substrate or as a regulator of biological processes. In certain embodiments, smaller macromolecules such as interfering RNA (RNAi) and microRNA (miRNA) molecules may be useful as candidate drugs and/or agents. In other embodiments, macromolecules including but not limited to proteins, peptides and fusion proteins may be useful as drugs and/or candidate drugs and/or drug agents.

Chemotherapy drug candidates are an important class of drug candidates and/or drug agents for the disclosed methods. Chemotherapeutic drug candidates include, without limitation, Carboplatin, Gemzar® (Gemocitabine), Taxol® (Paclitaxel), Metformin, Topotecan, Alimta® (Permetrexed), Abraxane, Barasertib, Everolimus, Cisplatin, Doxorubicin, Elesclomol, Irinotecan, Camptothecan, Olaparib, Etoposide, Vinorelbine, and/or a combination thereof.

Kinase inhibitors are an important class of drug candidates and/or drug agents for use in the disclosed methods. Kinase inhibitors interfere with the attachment of phosphate molecules to phosphorylation sites on target proteins. Many kinase inhibitors are commercially available (approved) and several are in clinical trials. Moreover, small molecules inhibitors of the activity of particular classes of protein kinases have become important anti-cancer drugs. (Ventura et al (2006) Clin Transl Oncol. 8: 153-60.) Approved kinase inhibitor cancer drugs include but are not limited to Vemurafenib, Lapatanib, Erlotinib, Imatinib. Such kinase inhibitors target kinases that are expressed intracellularly. Kinase inhibitors include, without limitation, Vemurafenib, Lapatinib, Gefitinib, Danusertib, Barasertib, Crizotinib, Pimasertib, Neratinib, Sorafenib, Selumetinib, monoclonal antibodies (e.g., Ipilumumab), DTIC, taxane analogs (e.g., abraxane, Taxol®, taxotere), and/or a combination thereof.

Biologics are a class of drug synthesized or derived from a living organism and may be used as a candidate drug or drug agent. For example, an antibody preparation such as Herceptin® (Trastuzumab) is considered a biologic.

Other small molecule drugs include, without limitation, protease inhibitor Bortezomib, PARP (poly ADP ribose polymerase) inhibitors Rucaparib and Olaparib, hypomethylating agent Dacogen (Decitabine), DNA damage inducer Methazolastone, alkylating agent Ifosfamide, and/or a combination thereof.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DEFINITIONS

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482.

A “macromolecule” means a large biological polymer including but not limited to nucleic acids, proteins, carbohydrates and lipids.

“Cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Antibody” refers to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

“Administration” and “treatment” or “therapy” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” or “therapy” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment” or “therapy” as it applies to a human, veterinary, or research subject, refers to therapeutic treatment, prophylactic or preventative measures, to research and diagnostic applications. “Treatment” or “therapy” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts a receptor (or heterodimer), e.g., in the fluid phase or colloidal phase, but also situations where the agonist or antagonist does not contact the cell or the receptor.

“Patient” includes humans and non-human mammals (e.g., monkeys, dogs, cats, rabbits, cattle, horses, sheep, goats, swine, and the like).

“Inhibits” means measurably slows, decreases, interferes with or stops or blocks enzymatic activity. Desirably, a slowing or decrease of the enzymatic activity is by at least 20%, 30%, 50%, 70%, 90%, or even 100% as determined using a suitable assay for measuring of enzymatic activity.

“Isolated nucleic acid molecule” or “isolated protein” or “isolated antibody” refers to a nucleic acid molecule or protein or antibody that is identified and separated from at least one contaminant nucleic acid, protein or antibody molecule with which it is ordinarily associated in the natural source. An isolated nucleic acid molecule or protein or antibody is other than in the form or setting in which it is found in nature. Isolated nucleic acid molecules therefore are distinguished from the nucleic acid molecule as it exists in natural cells.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

A “nucleic acid molecule” means DNA or RNA. DNA molecule that is separated from sequences (or nucleotide sequences) with which it is immediately contiguous (in the 5′ and 3′ directions). For example, the “nucleic acid molecule” may comprise a DNA molecule inserted into a vector, such as a plasmid or virus vector, or integrated into the genomic DNA of a prokaryote or eukaryote. An “isolated nucleic acid molecule” may also comprise a cDNA (complementary DNA) molecule. An isolated nucleic acid molecule manipulated to include other nucleic acid sequences is often referred to as a recombinant molecule. An RNA molecule is composed of nucleotides (ribonucleotides) and is typically single-stranded. RNA is coded by the DNA molecule, or transcribed using the DNA molecule as a template, so that the messenger RNA (mRNA) can be translated into its corresponding amino acid sequence. Short interfering RNA is double-stranded RNA of about 20-25 base pairs (or nucleotides) in length, and which typically function to interfere with the expression of a gene or genes. MicroRNA (miRNA) are very small pieces of RNA which are about 22 nucleotides in length and typically function in the transcriptional or post-transcriptional regulation of a gene or genes.

An “amino acid molecule” means the protein or polypeptide encoded by the DNA molecule. Proteins are made up of one or more chains of amino acid sequences and have a wide variety of function in living cells and organisms.

The invention will now be described by way of Examples, which are meant to assist one of ordinary skill in the art in carrying out the invention and are not intended in any way to limit the scope of the invention.

### EXAMPLES

### Example 1

### Ex Vivo Proliferation Assay

Patient-derived tumor cells were allowed to thaw in 37° C. water bath until frozen cell suspension melts enough to form a slushy solution. The cell suspension was slowly transferred through a cell strainer to a 50 ml conical tube filled with conditioning media warmed to 37° C. Cells were pelleted by spinning the tubes at 800 RPM for 5 minutes at 4° C. The supernatant was carefully removed without disturbing the pellet by aspiration of the supernatant. The pellet was resuspended in 1-2 ml warmed conditioning medium. Cells were counted using TC10. 10 μl of the cell suspension was removed and transferred into a tube for Trypan Blue exclusion assay to assess the cell density and overall health of the cells in the vial. LIVE-DEAD cells were counted.

6-well plates were coated with Cultrex® (ECM from Trevigen). ECM was diluted 1:20 with ice cold conditioning medium without growth factors and supplements. 1 ml per well of 6-well plate or 3 ml per 100 mm dish of diluted Cultrex solution was added. Plate was incubated for 2 hours at 37° C. Coated plates were placed in the cell culture hood for 5-10 minutes at room temperature. Coating solution was carefully aspirated and cells were immediately seeded. The flask/dish with cells was transferred from the incubator to the microscope. Cells were observed under the microscope to determine the percentage of confluence. A rough estimate was assessed of the percentage of cells in the area of the flask/dish which is not covered by cells. Once cells reached 70% confluence, they were split/passaged.

Transfer media, Trypsin-EDTA solution and sterile PBS are warmed to 37° C. in a water bath and incubated for 15-30 minutes. Supernatant of the split/passaged cells was transferred to a 15-50 ml conical tube. 1 ml of sterile PBS per 1 well of 6-well plate or 5 ml per 100 mm dish was added to cover the monolayer. The monolayer was rinsed by tilting the flask/dish 2-3 times in both directions. The wash from the rinse is transferred to the 15-50 ml conical tube containing the supernatant. 1 ml per well of 6-well plate or 5 ml per 100 mm dish of Trypsin-EDTA solution (depending on the size of the culture vessel) was added to cover the monolayer. The flask/dish was incubated for 5-7 minutes in a CO₂ incubator. (PTCs grown on 1:20 ECM may need to be incubated with Trypsin for up to 10 minutes.) Cells were observed under the microscope. The sides of the flask/dish were gently tapped and the cells were observed detaching as single cells. An equal volume of medium containing FBS (Fetal Bovine Serum), also considered the Chemosensitivity Medium, was added as soon as the cells detached and were floating. (The FBS inactivates the trypsin and minimizes harm to the cells.) The cells were harvested and the entire contents of the flask/dish were transferred to a 50 ml conical tube. The tube was centrifuged at 200×G RPM for 5 minutes. The medium was carefully aspirated without disturbing the pellet. 1-2 ml of conditional medium was added and the pellet was resuspended by gently pipetting up and down 5 times. The cells were counted.

The cells were subcultured in 3D on 1:20 ECM. The cells were harvested from ECM by removing supernatant and tilting plate and not touching bottom of the plate with the pipette. The cells were rinsed with PBS. 1 ml Trypsin-EDTA was added per 1 well of 6-well plate or 5 ml per 10 cm dish. The cells were incubated for 5 minutes at 37° C. A scraper was used to complete lifting of the cells from the well and the chemosensitivity media was used to rinse the cells from the scraper to inactivate the trypsin. The flask/dish was returned to the incubator.

The drug stock solution was prepared by determining the chemical entity of the drug agent under use. Information was gathered based on the label (if obtained from a vendor) or information from sponsor. The concentration of the stock solution was prepared. The drug was incubated at room temperature (if stored at lower temperatures) for 30 minutes before opening the vial. The required amount of the drug is weighed. Required amount of DMSO (Dimethyl Sulfoxide) was added and mixed well to dissolve. 50-250 μl (depending on total volume mixed) was aliquotted and the storage tubes were labeled. Half of the stock of drug solution was stored at −80° C. The other half was split into two and one half is stored in −20° C. and the other half is stored at 2-8° C.

The working stock of the drug solution was prepared. The stock solution was diluted in appropriate cell culture medium to obtain 10 ml of 1 mM of working stock. The solution was filter sterilized through a 0.22 μm syringe filter.

The drug dilutions were prepared by diluting the drug 1 mM working stock or 1 μM stock (10 ml: prepared by diluting 1 mM stock 1000× in medium) in the cell culture medium. 1 ml of the drug dilution was added into the corresponding positions in a Nunc deep well plate and the appropriate quantities of drugs were added to the corresponding positions.

The cells were labeled with EdU (5-ethynyl-2′-deoxyuridine) by treating the cells with 10 μM EdU for 48 hours. The cells were fixed by determining the volume of fix solution (37% formaldehyde in PBS) required (100 μl per well). The fix solution was prepared by mixing 37% formaldehyde solution 1:10 with 1×PBS (prepare fresh every day). The plate with cells was removed from the incubator and 200 μl of medium was carefully removed from sides after tilting the plate 45-90°. 100 μl PBS at room temperature was added to each well. 80-90% of the liquids from each well were removed using 200 μl pipette. 200 μl of PBS was added and 80-90% of the liquids from each well were removed. 100 μl of fix solution was added and the plate was incubated in the dark for 15-20 minutes at room temperature. 100 μl of PBS was added and 80-90% of the liquids for each well were removed using a 200 μl pipette. Another 200 μl of PBS was added and the plate was incubated for 5-10 minutes in the dark. 80-90% of the liquids were removed. 100 μl of PBS was added.

Cells were permeabilized by determining the volume of the permeabilization solution (0.1% Triton X 100 in PBS) required (100 μl per well). Permeabilization solution was prepared by mixing 10 μl of Triton X 100 with 10 ml of 1×PBS (prepare fresh every day) and filtering through ha 1 μm syringe filter (non-sterile filter is OK). 100 μl permeabilization buffer was added after last wash. The plate was incubated for 15-20 minutes at room temperature. 100 μl of PBS was added and 80-90% of the liquids from each well were removed using a 200 μl pipette. Another 200 μl of PBS was added and the plate was incubated for 5-10 minutes. 80-90% of the liquids from each well were removed.

The cells were subjected to Click-iT® EdU staining by determining the total volume of Click-iT® HCS (high content screening) cocktail. Click-iT® EdU buffer additive was prepared by adding 2 ml distilled water to the bottle marked as Component E and mixing well. After first use, the buffer additive was stored at −20° C. 1× concentration of the buffer additive was prepared by diluting the 10× solution 1:10 in distilled water. 1× concentration of Click-iT® EdU reaction buffer was prepared by diluting 10× solution (Component C) 1:10 in distilled water. Alex Fluor Azide (Component B) was thawed. The components were mixed to prepare the cocktail (just before the assay) in the following order: 1× Click-iT® EdU reaction buffer (Component A), Copper Sulfate solution (Component D), Alexa Fluor Azide (Component B), and 1× Click-iT® EdU Buffer Additive (Component E); according to the following amounts:

1× Click-iT® EdU reaction buffer: 12 wells (640 μl), 24 wells (1.28 ml), 48 wells (2.55 ml), 96 wells (5.1 ml), 144 wells (7.7 ml), 192 wells (10.2 ml); Copper Sulfate solution: 12 wells (30 μl), 24 wells (60 μl), 48 wells (120 μl), 96 wells (240 μl), 144 wells (360 μl), 192 wells (4800); Alexa Fluor Azide: 12 wells (1.9 μl), 24 wells (3.75 μl), 48 wells (7.5 μl), 96 wells (15 μl), 144 wells (22.5 μl), 192 wells (30 μl); Click-iT® EdU Buffer Additive: 12 wells (75 μl), 24 wells (150 μl), 48 wells (300 μl), 96 wells (600 μl), 144 wells (900 μl), 192 wells (1.2 ml).

The plates were retrieved and the last wash was removed. 45-50 μl of the cocktail was added per well and the plates were incubated at room temperature in the dark for 30-60 minutes. The reaction cocktail was removed and 100 μl of Click-iT® reaction rinse buffer (stored at 4° C.) was added. The plates were incubated in the dark for 5-10 minutes. The rinse buffer was removed and 200 μl of PBS was added. The plates were incubated for 5-10 minutes in the dark at room temperature.

The cells were subjected to DAPI (4′,6′-Diamidino-2-Phenylindole, Dihydrochloride) (Nuclear) staining by determining the volume of DAPI solution required (50 μl per well). 1×DAPI solution was prepared by mixing 10 μl of stock solution in 10 ml of PBS. 50 μl of DAPI solution was added to each well and the cells were incubated at room temperature (protected from light) for 30 minutes. Excess DAPI was washed by adding 100 μl of PBS and 80-90% of the liquids from each well were removed using a 200 μl pipette. Another 200 μl of PBS was added and 80-90% of the liquids from each well were removed using a 200 μl pipette. 50 μl of PBS was added before analysis. The wells were read by HC imaging and the anti-proliferative efficacy of the drug was calculated.

### Example 2

### Use of Patient-Derived Xenografts (PDX)

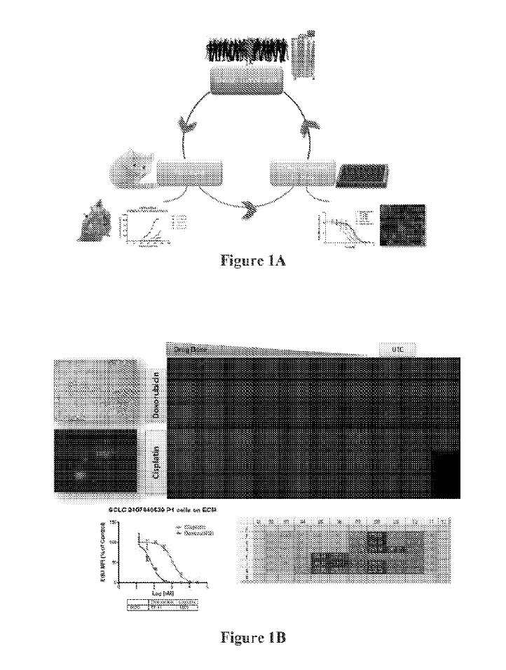

Fresh or banked patient-derived tumor cells were injected into immunocompromised mice. Cells were harvested and plated to form spheroids. Spheroids were subjected to ex vivo drug treatment using chemotherapy drug candidates, cisplatin and doxorubicin. Cell proliferation was measured by HC imaging by EdU incorporation assay. (FIG. 1A-B)

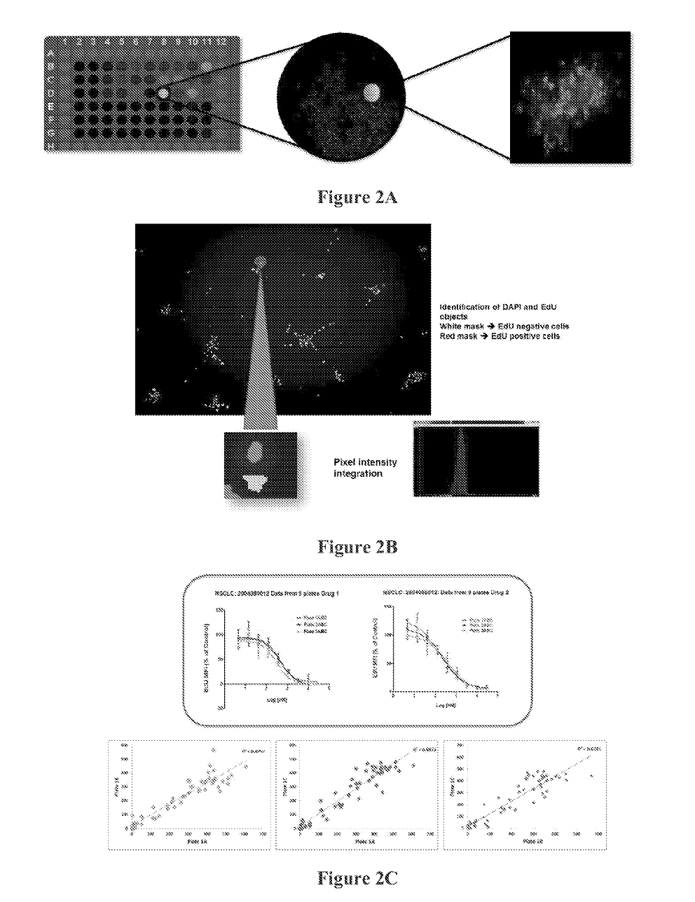

Cell images are captured and processed using multiple algorithms corresponding to the experiment. DAPI was selected on channel 1 to identify valid objects. EdU was selected on channel 2 to identify a population of cells incorporating DNA. Intensity for each pixel for each channel was recorded and integrated to obtain total intensity. Total intensity was divided by total pixels to give mean fluorescent intensity (MFI) for that channel. Integrated mean fluorescent intensity of multiple cells divided by total cells per well gave mean of MFI. To normalize the variability of cell numbers between wells, the mean of MFIs of EdU signals were used to assess in vitro drug effects. EdU MFIs were used to compare the drug effects across multiple plates. (FIG. 2A-C)

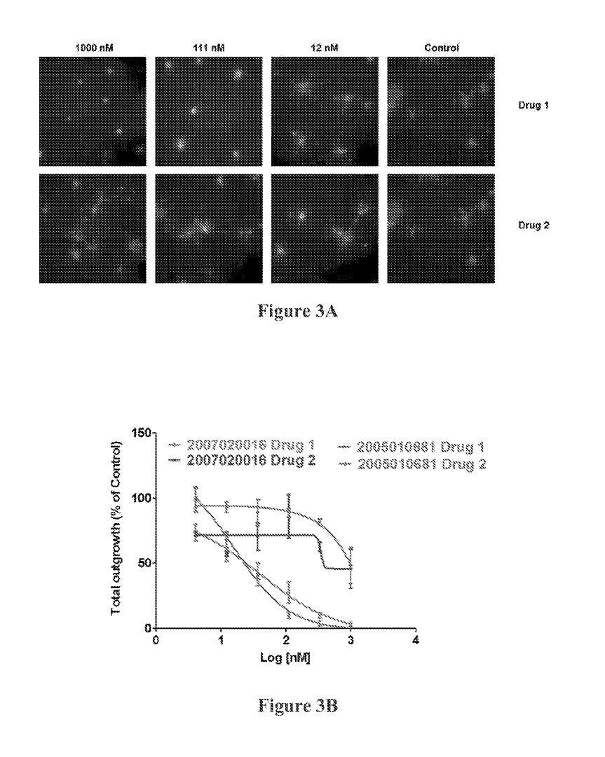

Spheroid morphology was used as a measure of drug effects on patient-derived tumor cells growing as spheroids. Staining for nucleus (DAPI), EdU (proliferating cell), and cytokeratin (CK) (epithelial cell) and HC imaging was conducted. The size of spheroid or tumor outgrowth was used to assess the effect of drug candidates that were chemotherapeutic agents. (FIG. 3A-C)

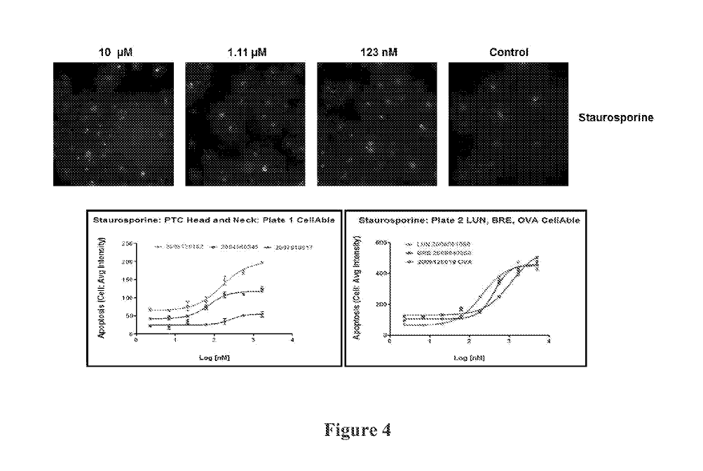

Other endpoints, such as apoptosis induction, were also used to evaluate effects of drug candidates such as staurosporine in PTCs of head and neck, lung, breast, and ovarian tumor cells. Apoptosis (Cell: Average intensity) induction was measured using CellEvent™ and quantified using HC imaging. (FIG. 4)

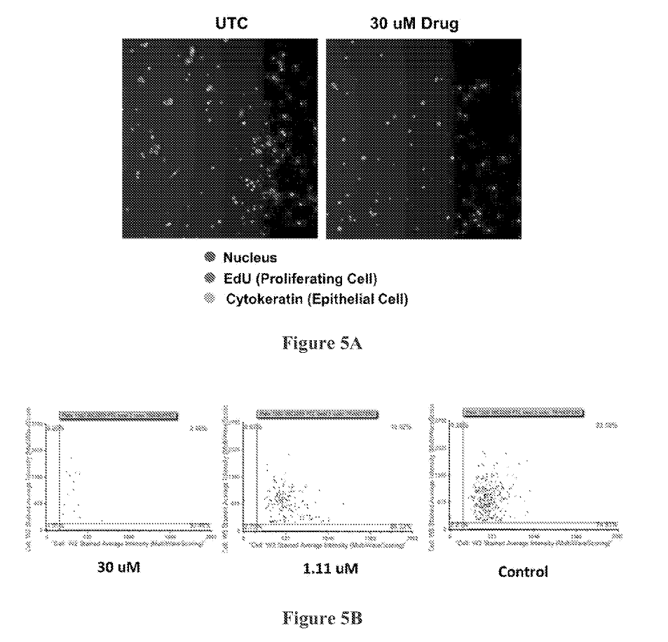

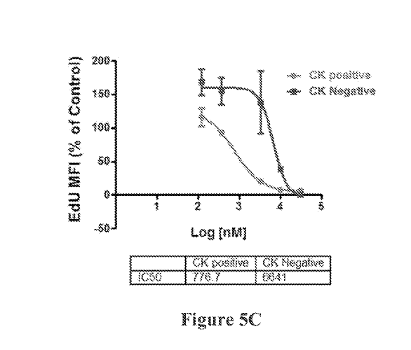

Subpopulation Analysis of tumor tissue was conducted. By combining the EdU incorporation assay with staining for tumor specific markers, effects of drug candidates in specific populations of cells was assessed. CK was used as a marker for epithelial cells (mostly tumor cells). Fluorescent images of spheroids were analyzed using image cytometry algorithms to look at the proliferation in subsets of cells and analyzed for drug effects of interest. (FIG. 5A-C)

All publications mentioned in the above specification are herein incorporated by reference. Various modifications and variations of the described methods will be apparent to those skilled in the art without departing from the scope and spirit of the invention. Although the invention has been described with respect to particular aspects and/or further embodiments, it should be understood that the invention as claimed should not be unduly limited to such aspects and/or embodiments. It should also be understood that various modifications of the described modes for carrying out the invention, which would be readily known to and/or accessed through available information by those skilled in cellular studies or related fields, are intended to be within the scope of the following claims.

What is claimed is:

1. A method of testing proliferative responses of a drug on patient-derived tumor cells; the method comprising:
a. obtaining cells from biopsy or tumor resection material;
b. culturing the cells on a 3D extracellular matrix (ECM);
c. treating the cells in ECM with a drug;
d. subjecting the treated cells to high-content (HC) imaging; and
e. evaluating the HC imaging of the treated cells; thereby testing the proliferative responses of the drug on the patient-derived tumor cells.

2. The method of claim 1, wherein the treated cells are in the formation of a tumor spheroid.

3. The method of claim 1, wherein the biopsy or tumor resection material is from a biobank.

4. The method of claim 1, wherein the patient-derived tumor cells are primary tumor cells (PTCs).

5. The method of claim 1, wherein the patient-derived tumor cells are selected from the group consisting of breast cancer cells, prostate cancer cells, non-small cell lung cancer cells, ovarian cancer cells, melanoma cells, and pancreatic cancer cells.

6. The method of claim 1, wherein the drug is selected from the group consisting of small molecule drugs, kinase inhibitors, macromolecules, and a combination thereof.

7. The method of claim 1, wherein the proliferative responses are selected from the group consisting of EdU incorporation, LIVE-DEAD cell counts, colony formation, and a combination thereof.

8. The method of claim 1, wherein the treated cells are evaluated by techniques selected from the group consisting of proliferation, colony morphology, apoptosis, and a combination thereof.

9. A method of testing proliferative responses of a drug on patient-derived tumor cells; the method comprising:
a. obtaining cells from biopsy or tumor resection material;
b. xenografting the cells into a mouse for tumor formation;
c. obtaining tumor cells from the mouse;
d. culturing the tumor cells on a 3D extracellular matrix (ECM);
e. treating the tumor cells in ECM with a drug;
f. subjecting the treated tumor cells to high-content (HC) imaging; and
g. evaluating the HC imaging of the treated cells; thereby testing the proliferative responses of the drug on the patient-derived tumor cells.

10. The method of claim 1, wherein the treated tumor cells are in the formation of a tumor spheroid.

11. The method of claim 1, wherein the biopsy or tumor resection material is from a biobank.

12. The method of claim 1, wherein the patient-derived tumor cells are primary tumor cells (PTCs).

13. The method of claim 1, wherein the patient-derived tumor cells are selected from the group consisting of breast cancer cells, prostate cancer cells, non-small cell lung cancer cells, ovarian cancer cells, melanoma cells, and pancreatic cancer cells.

14. The method of claim 1, wherein the drug is selected from the group consisting of small molecule drugs, kinase inhibitors, macromolecules, and a combination thereof.

15. The method of claim 1, wherein the proliferative responses are selected from the group consisting of EdU incorporation, LIVE-DEAD cell counts, colony formation, and a combination thereof.

16. The method of claim 1, wherein the treated tumor cells are evaluated by techniques selected from the group consisting of proliferation, colony morphology, apoptosis, and a combination thereof.
